(19) United States
(12) Patent Application Publication
(10) Pub. No.: US 2010/0330098 A1
(43) Pub. Date: Dec. 30, 2010

(54) METHODS TO REGULATE GLUCOSE METABOLISM

(76) Inventors: Calvin J. Kuo, Palo Alto, CA (US); Kevin Wei, Stanford, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/825,602

(22) Filed: Jun. 29, 2010

Related U.S. Application Data
(60) Provisional application No. 61/221,189, filed on Jun. 29, 2009.

(51) Int. Cl. A61K 39/395; A61K 31/7088; C07K 16/00; C07H 21/04; A61P 3/10; C12N 5/071

(52) U.S. Cl. 424/158.1; 424/130.1; 424/172.1; 514/44 R; 514/44 A; 530/387.1; 530/389.1; 530/389.2; 536/23.1; 536/24.5; 435/375

(57) ABSTRACT

The present disclosure describes an unexpected and novel insight into hepatic insulin resistance through the description of the ability of VEGF inhibitors to revert hyperglycemia and hyperinsulimenia in murine Type 2 diabetes mellitus models through modulation of a HIF-2α-IRS-2 axis operative in hepatocytes. As such, the data of the present disclosure uncover a novel pathway regulating hepatic IRS-2 expression, and identify VEGF inhibitors as an FDA-approved class of therapeutics capable of increasing liver IRS levels and ameliorating Type 2 diabetes mellitus. Accordingly, this disclosure identifies stabilization of HIF-2α as a robust method for improving glucose metabolism. The disclosure provides embodiments of a method of modulating the level of glucose metabolism of a mammalian cell, comprising: contacting a mammalian cell with an effective amount of a composition, where the composition can modulate the levels of HIF-2α and IRS-2 activity in said cell, thereby modulating the level of glucose metabolism by said cell. The disclosure also provides embodiments of a pharmaceutical composition and methods for their use for the treatment of Type 2 diabetes in an animal or human subject, the method comprising administering to an animal or human subject a effective amount of a pharmaceutically acceptable composition, the composition, when administered to an animal or human subject, increasing the activity of HIF-2α and IRS-2 in the animal or human subject, thereby reducing the level of blood glucose in the animal or human subject.

### CROSS-REFERENCE TO RELATED APPLICATIONS

This application claims priority to U.S. Provisional Patent Application Ser. No. 61/221,189, entitled “METHODS TO INCREASE ENDOGENOUS LEVELS OF IRS-2” filed on Jun. 29, 2009, the entirety of which is hereby incorporated by reference.

### TECHNICAL FIELD

The present disclosure is generally related to methods of modulating endogenous levels of glucose in an isolated cell or a whole animal. The present disclosure further relates to methods of modulating the symptoms of Type 2 diabetes mellitus in a patient by modulating endogenous levels of HIF proteins and IRS-2.

### BACKGROUND

Type 2 diabetes mellitus is a leading cause of kidney failure, blindness, and amputations, and is a major risk factor for heart disease and stroke (Saltiel & Kahn (2001) Nature 414: 6865). The central pathophysiologic defect of Type 2 diabetes mellitus is insulin resistance, where tissues such as muscle, fat and liver exhibit reduced responsiveness to insulin, despite normal to elevated insulin levels.

Emerging data from animal and human studies strongly suggests that insulin resistance in liver is the key lesion in the development of Type 2 diabetes mellitus (Michael et al., (2000) Mol. Cell 6: 87; Tripathy et al., (2004) Diabetologia 47: 782; Kim et al., (2003) Diabetes 52: 2453). Insulin decreases blood glucose by actively suppressing hepatic glucose production through hepatic insulin receptors that utilize the adapter protein insulin receptor substrate (IRS) proteins to activate Akt kinase. Insulin-induced Akt phosphorylation of the FOXO1 transcription factor leads to FOXO1 exclusion from the nucleus, with a consequent decreased expression of the FOXO1-targeted genes glucose 6-phosphatase and phosphoenolpyruvate carboxylase that are the rate-limiting enzymes of gluconeogenesis. Other mechanisms for insulin-regulated hepatic glucose homeostasis are decreased transcription of the glycolytic genes glucokinase, pyruvate kinase, and increased gluconeogenic substrates via PKA/cAMP antagonism.

In Type 2 diabetes mellitus, therefore, hepatic insulin resistance results in increased hepatic glucose production since insulin is unable to repress glucose 6-phosphatase and phosphoenolpyruvate carboxylase levels, yielding higher fasting and fed blood glucose levels (Michael et al., (2000) Mol. Cell 6: 87). This exacerbates an already deleterious situation of hyperglycemia and chronic hyperinsulinemia (Tripathy et al., (2004) Diabetologia 47: 782; Kim et al., (2003) Diabetes 52: 2453). Hepatic insulin resistance further increases fatty acid and triglyceride synthesis, leading to a cycle of hepatic steatosis and further insulin resistance (Shimomura et al., (2000) Mol. Cell 6: 77). Furthermore, pancreatic attempts to compensate through increasing insulin production can culminate in “islet cell exhaustion” with β-cell failure in end-stage Type 2 diabetes mellitus patients.

It has been postulated that pharmacologic modulation of IRS proteins could be of therapeutic utility for diabetes treatment (Lee & White (2004) Arch. Pharm. Res. 27: 361). IRS proteins carry out various functions downstream of the insulin receptor by providing a juxtamembrane localization signal for phosphatidylinositol (3,4,5)-trisphosphate (PIP3) generation, and amplifying the signal engendered by receptor autophosphorylation (Haeusler & Accili (2008) Cell Metab. 8: 7). IRS-2 expression is regulated both transcriptionally and post-transcriptionally, and can be stimulated by hormonal signals (White, M. F., (2003) Science 302: 1710; White, M. F., (2002) Am. J. Physiol. Endocrinol. Metab. 283: E413; Jhala et al., (2003) Genes Dev. 17: 1575). Notably, in the insulin-resistant db/db mouse model of Type 2 diabetes the liver is strongly deficient in IRS-2, and restoration of IRS-2 to db/db liver via adenoviral over-expression in vivo restores insulin sensitivity and reverts the db/db mouse diabetic phenotype (Canettieri et al., (2005) Cell Metab. 2: 331-338). Restoration of hepatic IRS-2 is, therefore, been considered an attractive potential strategy for Type 2 diabetes mellitus treatment, although pharmacologic agents capable of inducing IRS-2 expression have been sorely lacking (White, M. F., (2003) Science 302: 1710; White, M. F., (2002) Am. J. Physiol. Endocrinol. Metab. 283: E413).

### SUMMARY

The pathophysiology underlying insulin resistance in Type 2 diabetes mellitus is of critical relevance to the development of effective treatments for this disorder. An increasing body of evidence has implicated hepatic insulin resistance and association with hepatic IRS-2 deficiency as particularly significant to Type 2 diabetes mellitus pathogenesis, with genetic restoration of liver IRS-2 expression being sufficient to treat mouse Type 2 diabetes mellitus models.

The present disclosure describes an unexpected and novel insight into hepatic insulin resistance through the description of the ability of VEGF inhibitors to revert hyperglycemia and hyperinsulimenia in murine Type 2 diabetes mellitus models through modulation of a HIF-2α-IRS-2 axis operative in hepatocytes. As such, the data of the present disclosure uncover a novel pathway regulating hepatic IRS-2 expression, and identify VEGF inhibitors as an FDA-approved class of therapeutics capable of increasing liver IRS levels and ameliorating Type 2 diabetes mellitus.

One aspect of the disclosure provides embodiments of a method of modulating the level of glucose metabolism of a mammalian cell, comprising: contacting a mammalian cell with an effective amount of a composition, where the composition can modulate the levels of HIF-2α and IRS-2 activity in said cell, thereby modulating the level of glucose metabolism by said cell.

In embodiments of this aspect of the disclosure, the modulation of the HIF-2α level in the mammalian cell increases the level of IRS-2 activity in the cell, thereby decreasing the level of activity of at least one gluconeogenic enzyme of the cell.

In embodiments of this aspect of the disclosure, the modulation of the HIF-2a level in the mammalian cell increases the level of IRS-2 activity in the cell, thereby increasing the level of activity of at least one glycolytic enzyme of the cell.

In embodiments of this aspect of the disclosure, an increase in HIF-2α activity in the mammalian cell can increase the level of IRS-2 activity in the cell.

In some embodiments of this aspect of the disclosure, the composition modulating the level of HIF-2α in the cell modulates the interaction of VEGF with the cell.

In some embodiments of this aspect of the disclosure, the composition reduces the interaction of VEGF and the cell compared to when the composition is not in contact with the cell.

In embodiments of this aspect of the disclosure, the composition is a VEGF antagonist. In these embodiments of this aspect of the disclosure, the composition can be a VEGF antagonist selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In other embodiments of this aspect of the disclosure, the composition generates an hypoxic response mimetic in the mammalian cell, thereby increasing the level of HIF-2α in the cell.

In embodiments of this aspect of the disclosure, the mammalian cell can be an hepatic cell.

In embodiments of this aspect of the disclosure, the mammalian cell can be an isolated cell or a population thereof.

In embodiments of this aspect of the disclosure, the mammalian cell can be in a tissue of an animal or human, and wherein the level of glucose in the blood of the recipient animal or human is reduced.

Another aspect of the disclosure provides embodiments of a method for the treatment of Type 2 diabetes in an animal or human subject, the method comprising administering to an animal or human subject a effective amount of a pharmaceutically acceptable composition, the composition, when administered to an animal or human subject, increasing the activity of HIF-2α and IRS-2 in the animal or human subject, thereby reducing the level of blood glucose in the animal or human subject.

In embodiments of this aspect of the disclosure, the pharmaceutically acceptable composition comprises an antagonist of VEGF activity in the subject.

In these embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In some embodiments of this aspect of the disclosure, the pharmaceutically acceptable composition can generate an hypoxic response mimetic in the subject animal or human and comprises an inhibitor of a prolyl hydroxylase activity.

In embodiments of this aspect of the disclosure, the modulation of the HIF-2α level in the cell can increase the level of IRS-2 activity, thereby decreasing the level of activity of at least one gluconeogenic enzyme.

In these and other embodiments of this aspect of the disclosure, the modulation of the HIF-2α level in the cell increases the level of IRS-2 activity, thereby increasing the level of activity of at least one glycolytic enzyme.

Still another aspect of the disclosure provides embodiments of pharmaceutical composition comprising an effective dose of a therapeutic agent that when administered to a animal or human subject induces an increase in the level of HIF-2α activity in the cell, thereby decreasing the blood glucose level of the recipient subject.

In embodiments of this aspect of the disclosure, the pharmaceutical composition, when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

In embodiments of this aspect of the disclosure, the therapeutic agent can be a VEGF antagonist in an amount that when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

In embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In other embodiments of this aspect of the disclosure the therapeutic agent can generate an hypoxic response mimetic in the subject animal or human and comprises an inhibitor of a prolyl hydroxylase activity.

Yet another aspect of the disclosure provides embodiments of an article of manufacture comprising a package containing a pharmaceutical composition that when administered as an effective dose to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased, where the pharmaceutical composition can comprise a therapeutic agent that is a VEGF antagonist or an hypoxic response mimetic, said mimetic comprising an inhibitor of a prolyl hydroxylase activity.

In embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIG. 1 shows ad libidum and fasting blood glucose levels of 10-12 week-old C57Bl/6 (n=7) mice two weeks following intravenous injection with recombinant Ad Flt1 (encoding a soluble VEGF receptor) to induce VEGF inhibition, or the control virus Ad Fc, at 5×10⁸ pfu. Note decreased blood glucose with VEGF inhibition.

FIG. 2 shows a graph of blood glucose levels of 10-12 week-old SCID (n=5) mice two weeks after twice-weekly subcutaneous treatment with 40 mg/kg anti-VEGFR2 mAb (clone DC101) or control rat IgG. Decreased ad libidum and fasting blood glucose levels are seen with DC101 vs. control.

FIG. 3A shows a graph illustrating the results of a glucose tolerance test (GTT). Adult C57Bl/6 mice (n=5) received single intravenous injections of 5×10⁸ pfu Ad Flt1 or Ad Fc (FIG. 3A). 15 days later, mice were fasted for 16 h and given 2 mg/kg glucose intraperitoneally and blood glucose levels were measured. * P≦0.04 vs. control-treated animals at the indicated time points.

FIG. 3B shows a graph illustrating the results of a glucose tolerance test (GTT). Adult SCID mice (n=5) were treated with VEGF Trap twice weekly with 25 mg/kg subcutaneously. 15 days later, mice were fasted for 16 h and given 2 mg/kg glucose intraperitoneally and blood glucose levels were measured. * P≦0.04 vs. control-treated animals at the indicated time points.

FIG. 4 is a Western blot indicating increased hepatic insulin signaling following single intravenous injections of 5×10⁸ pfu Ad Flt1 (VEGF inhibition) or Ad Fc (negative control) into adult C57Bl/6 mice. Ad Flt1, but not Ad Fc, increases phosphorylation of the insulin receptor (IR) signaling intermediates Akt (to give p-Akt) and GSK-3β (to give p-GSK-3β). Liver IRS-2 protein levels were also increased by Ad Flt1. IR-β: b-subunit of the IR. * P≦0.05; n=5 animals each.

FIG. 5 is a graph showing decreases in hepatic gluconeogenic gene expression following single intravenous injections of 5×10⁸ pfu Ad Flt1 (VEGF inhibition) or Ad Fc (negative control) into adult C57Bl/6 mice, again consistent with enhanced hepatic insulin signaling upon VEGF inhibition. qPCR data is shown from liver RNA, * P≦0.05; n=5 animals each.

FIG. 6 shows a graph of blood glucose levels 8-12 week-old SCID (n=8) mice 6 weeks after twice-weekly subcutaneous treatment with recombinant VEGF Trap (a soluble form of VEGF receptor) or control human Fc at the indicated doses. * P<0.05 for control vs. treatments.

FIG. 7A shows a graph of the glucose infusion rate as determined by euglycemic, hyperinsulinemic clamp analyses. Adult male C57Bl/6 mice (n=8 or 9) were used. * P=0.01 ** P=0.0001 for control vs. treatment.

FIG. 7B shows a graph of the insulin suppression of hepatic glucose production as determined by euglycemic, hyperinsulinemic clamp analyses. Adult male C57Bl/6 mice (n=8 or 9) were used. * P=0.01 ** P=0.0001 for control vs. treatment.

FIG. 8 is a digital image of a Western blot analysis.

FIG. 9 shows a graph of a real-time quantitative PCR analysis of liver extracts from 10-12 weeks old SCID mice (n=3 or 5) six weeks after twice-weekly subcutaneous treatment with VEGF Trap or human Fc at 25 mg/kg. Values are mean±s.e.m.

FIG. 10 is a digital image of a heat map representation of selected HIF target gene expressions from livers of 10-12 week-old SCID mice (n=4 or 5) treated with twice-weekly subcutaneous injections of 25 mg/kg VEGF Trap or control hFc for 8 weeks.

FIG. 11 is a digital image of immunoblots of hepatic HIF-1α and HIF-2α from 10-12 week-old C57Bl/6 mice (n=3) 14 days after single injection of 5×10⁸ pfu of Ad Flt1 or control Ad Fc, revealing selective stabilization of HIF-2α but not HIF-1α.

FIG. 12 shows a graph showing ad libidum (20 d) and fasting (21 d) blood glucose levels after adenoviral infection. * P≦0.05 between Ad Fc and Ad Flt1 treated HIF-2α^{loxP/loxP}; albumin-Cre mice. Here, the decreased plasma glucose with Ad Flt1 (VEGF inhibition) is reversed by simultaneous HIF-2α LKO (liver-specific knock-out) in HIF-2α^{loxP/loxP}; albumin-Cre mice.

FIGS. 13A and 13B illustrate graphs showing the results of glucose tolerance tests. 21 d after adenoviral infection, mice were given 2 g/kg glucose intraperitoneally and blood glucose levels were measured. * P=0.03 and ** P=0.01 between Ad Fc- and Ad Flt1-treated mice at the indicated time points. The glucose tolerance with Ad Flt1 (VEGF inhibition) is wild type (WT) mice (FIG. 13A) is largely abrogated by simultaneous HIF-2α LKO in HIF-2α^{loxP/loxP}; albumin-Cre mice (FIG. 13B).

FIG. 14 is a digital image of a Western blot analysis of liver extracts of wild-type mice treated with the indicated Ad Fc and Ad Flt1 adenovirus constructs in vivo. The induction of IRS-2 by Ad Flt1 is then abrogated by the simultaneous HIF-2α LKO in HIF-2α^{loxP/loxP}; albumin-Cre mice.

FIG. 15 is a graph showing decreased ad libidum and fasting blood glucose levels with HIF-2α activation (i.e. HIF-2αPN) vs. Ad Fc (negative control). *: P≦0.05 between control and treatment groups.

FIG. 16 is a graph showing the results of a glucose tolerance test. Mice were given 2 g/kg glucose intraperitoneally and blood glucose levels were measured. Improved glucose tolerance with Ad HIF-2αPN (i.e. liver-specific HIF-2α activation) is evidenced by lower plasma glucose levels during the glucose tolerance test vs. the control Ad Fc. *: P<0.01 between treatment groups at the indicated time points.

FIG. 17 is a digital image of a Western blot analysis of liver extracts from Ad HIF-2αPN—(i.e. constitutively active mutant form of HIF-2α) vs. Ad Fc-(negative control) treated mice indicating that HIF-2α activates IRS-2 expression as well as downstream AKT and FOXO1 phosphorylation. Robust induction of liver IRS-2 by Ad HIF-2αPN, but not by Ad Fc, is observed (see arrow).

FIG. 18 is a graph showing a quantitative PCR analysis of the repression of the insulin-suppressed genes g6pc and pepck by Ad HIF-2αPN, but not Ad Fc. This is consistent with HIF-2α activation of insulin signaling in vivo. n=5 mice.

FIG. 19 is a digital image of a Western blot analysis of mouse primary hepatocytes transduced with Ad HIF-2αPN or Ad Fc and followed by insulin stimulation or vehicle treatment. HIF-2αPN strongly induced IRS-2 expression in primary hepatocytes. Further, HIF-2αPN and insulin strongly synergize to induce Akt phosphorylation (p-Akt), indicating that HIF-2α is sufficient to sensitize hepatocytes to insulin signaling in vitro.

FIG. 20 is a digital image of a Western blot analysis of mouse primary hepatocytes from the same experiment as illustrated in FIG. 19, except for shorter exposure of the p-Akt lane and the inclusion of the additional condition of infection with adenovirus encoding a shRNA targeting IRS-2 (Ad IRS-2 shRNA). The Ad IRS-2 shRNA abrogates the induction of IRS-2 by Ad HIF-2αPN, and blunts the HIF-2α/insulin synergistic phosphorylation of Akt (p-Akt), indicating that HIF-2α sensitization of hepatic insulin signaling requires IRS-2.

FIG. 21 is a graph showing the results of a quantitative PCR experiment demonstrating robust induction of IRS-2 mRNA upon infection of primary mouse hepatocytes with Ad HIF-2αPN but not with Ad Fc.

FIG. 22 schematizes the synergistic activation of the insulin receptor (IR) signaling pathway by HIF-2α and insulin.

FIG. 23 is a graph showing luciferase activity in hepatic 293T cells transfected with an IRS-2 promoter/luciferase reporter gene and HIF-2αPN, or in the presence of the hypoxia-mimetic Deferoxamine (DFO), indicating that the IRS-2 promoter is inducible by HIF-2α or hypoxia. * P<0.05 for treatment groups compared to control.

FIG. 24 is a graph showing mouse primary hepatocytes incubated in a hypoxia chamber at the indicated O₂ concentration for 24 h followed by real-time PCR quantification of IRS-2-specific mRNA, indicating that the endogenous IRS-2 gene is hypoxia-responsive. * P<0.05 compared to control (16% O₂).

FIG. 25 is a graph showing ad libidum and fasting blood glucose levels in mice. * P≦0.05 in the indicated treatment groups compared to control. Ad HIF-2αPN (i.e. HIF-2α activation) and Ad IRS-2 (i.e. restoration of IRS-2 to the IRS-2-deficient db/db liver) are both sufficient to phenocopy the Ad Flt1 reversal of the diabetic hyperglycemia of db/db, mice.

FIG. 26 is a graph showing the results of glucose tolerance tests. 7 d after adenoviral infection, mice were given 0.5 g/kg glucose intraperitoneally and blood glucose levels were measured. Again, Ad HIF-2αPN (i.e. HIF-2α activation) and Ad IRS-2 (i.e. restoration of IRS-2 to the IRS-2-deficient db/db liver) are both sufficient to phenocopy the Ad Flt1 reversal of the diabetic hyperglycemia of db/db mice. * P<0.05 between treatment groups and control at the indicated time points.

FIG. 27 is a digital image of a Western blot analysis of liver extracts showing robust restoration of hepatic IRS-2 expression and increased Akt phosphorylation by Ad HIF-2αPN but not Ad Fc treatment of db/db mice. The deficiency of IRS-2 in control (i.e. Ad Fc)-treated db/db mice vs. higher levels in db/+ controls is consistent with prior studies indicating that db/db liver is IRS-2-deficient.

FIG. 28 is a graph showing ad libidum blood glucose levels. * P<0.04 between VEGF Trap- and hFc-treated db/db mice at the indicated time points.

FIG. 29 is a graph showing the results of a glucose tolerance test in db/db mice. Mice were given 0.5 g/kg glucose intraperitoneally and blood glucose levels were measured. * P<0.03 between treatment groups at the indicated time points. Values are mean±s.e.m.

FIG. 30 is a graph showing the results of a glucose tolerance test in db/db mice. Mice were given 0.5 g/kg glucose intraperitoneally and blood glucose levels were measured. * P<0.03 between treatment groups at the indicated time points. Values are mean±s.e.m.

FIG. 31 is a graph showing plasma insulin levels as determined by ELISA. * P=0.003 between VEGF Trap- and hFc-treated db/db mice. The hyperinsulinemia of db/db mice is corrected by VEGF Trap treatment.

FIG. 32 is a graph showing the results of a glucose tolerance test in db/db mice. Mice were given 0.5 g/kg glucose intraperitoneally and blood glucose levels were measured. * P<0.02 between treatment groups at the indicated time points. Values are mean±s.e.m. The marked improvement of glucose tolerance with Ad Flt1 (i.e. VEGF inhibition) is abrogated by the simultaneous expression of the HIF-2α shRNA, indicating that VEGF inhibitors require HIF-2α to correct glucose intolerance in db/db mice.

FIG. 33 is a graph showing the fed and fasting blood glucose levels in db/db mice. As above, the marked improvement in fed and fasting blood glucose levels with Ad Flt1 (i.e. VEGF inhibition) is abrogated by the simultaneous expression of the HIF-2α shRNA, indicating that VEGF inhibitors require HIF-2α to correct glucose intolerance in db/db mice. * P<0.01, #P<0.04,

FIGS. 34A and 34B illustrates a schematic model for the reversal of hepatic insulin resistance by VEGF inhibitors.

FIG. 34A is a schematic illustrating the deficiency in IRS-2 rendering liver hepatocytes in db/db mouse liver insulin-resistant. Insulin signaling fails to suppress glucose production resulting in hyperglycemia and secondary hyperinsulinemia.

FIG. 34B is a schematic illustrating that VEGF inhibition stabilizes hepatic HIF-2α which transactivates and then induces endogenous IRS-2 expression. IRS-2 induction leads to the amplification of insulin receptor downstream signaling, Akt and FOXO1 phosphorylation, FOXO1 nuclear exclusion, and loss of G6PC/PEPCK expression, thereby suppressing excess glucose production and correcting hyperglycemia and hyperinsulinemia.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

The term ‘diabetes’ as used herein refers to abnormally high blood sugar levels due to defects in either insulin secretion or insulin action in the body. The term Type 1 diabetes has universally replaced several former terms, including childhood-onset diabetes, juvenile diabetes, and insulin-dependent diabetes mellitus (IDDM). The term Type 2 diabetes has replaced several former terms, including adult-onset diabetes, obesity-related diabetes, and non-insulin-dependent diabetes mellitus (NIDDM).

Type 1 diabetes mellitus is characterized by loss of the insulin-producing beta cells of the islets of Langerhans in the pancreas, leading to a deficiency of insulin. This type of diabetes can be further classified as immune-mediated or idiopathic.

Type 2 diabetes mellitus is characterized differently and is due to insulin resistance or reduced insulin sensitivity, combined with relatively reduced insulin secretion which in some cases becomes absolute. In the early stage of Type 2 diabetes, the predominant abnormality is reduced insulin sensitivity, characterized by elevated levels of insulin in the blood. At this stage hyperglycemia can be reversed by a variety of measures and medications that improve insulin sensitivity or reduce glucose production by the liver. As the disease progresses, the impairment of insulin secretion worsens, and therapeutic replacement of insulin often becomes necessary.

Both lead to hyperglycemia, which largely causes the acute signs of diabetes: excessive urine production, resulting compensatory thirst and increased fluid intake, blurred vision, unexplained weight loss, lethargy, and changes in energy metabolism. All forms of diabetes have been treatable since insulin became medically available in 1921, but there is no cure. The injections by a syringe, insulin pump, or insulin pen deliver insulin, which is a basic treatment of Type 1 diabetes. Type 2 diabetes is managed with a combination of dietary treatment, exercise, medications and insulin supplementation.

Diabetes and glucose regulation can be monitored by techniques known in the art, including a fasting plasma glucose test, an oral glucose tolerance test, a two-hour postprandial plasma glucose, and the like. Such determinations of diabetes rely on abnormalities in insulin levels or insulin function.

A fasting plasma glucose test is a carbohydrate metabolism test that measures plasma, or blood, glucose levels after a 12-14 hour fast period. Fasting stimulates the release of the hormone glucagon, which in turn raises plasma glucose levels. In non-diabetic individuals the body will produce and process insulin to counteract the rise in glucose levels. In diabetics this does not happen, and the tested glucose level remains high. The “normal” range for results may vary according to the lab procedures used.

A glucose tolerance test is a test that measures blood glucose levels four to five times over a 3-hour period. The subject animal or human is administered an oral or intraperitoneal dose of glucose solution, which should cause glucose levels to rise in the first hour, and then fall back to normal within three hours as the body produces insulin to normalize glucose levels. Typically, for mice the glucose is delivered intraperitoneally, which is apparently more specific for liver effects. Glucose levels that quickly rise above normal levels and take longer to normalize usually indicate diabetes mellitus.

The term “VEGF inhibitor” as used herein refers to a substance including, but not limited to, proteins, peptides, and modified variants thereof, that can selectively bind to VEGF or a VEGF receptor, thereby blocking binding of VEGF to a VEGF-specific receptor, and which retards or prevents a chemical or physiological reaction or response. Common blockers or inhibitors include, but are not limited to, antisense molecules, antibodies, antagonists and their derivatives. More specifically, an example of a VEGF blocker or inhibitor is a VEGF receptor-based antagonist including, but not limited to, an anti-VEGF antibody, or a VEGF Trap antagonist such as described in PCT publication WO/00/75319, the contents of which is herein incorporated by reference in its entirety.

In some embodiments of the disclosure, the VEGF antagonist can be a dimeric protein capable of binding VEGF with high affinity and composed of two receptor-Fc fusion polypeptides consisting of the principal ligand-binding portions of the human VEGFR1 and VEGFR2 receptor extracellular domains fused to the Fc portion of human IgG1 (the “VEGF Trap”). Specifically, the VEGF “trap” is the Ig domain 2 from VEGFR1 fused to Ig domain 3 from VEGFR2, which in turn is fused to the Fc domain of IgG1. Since the VEGF trap binds its ligands using the binding domains of high-affinity receptors, it has a greater affinity for VEGF than do monoclonal antibodies.

The term “pharmaceutically” or “pharmaceutically acceptable” as used herein refer to molecular entities and compositions that do not produce an adverse, allergic or other untoward reaction when administered to an animal, or a human, as appropriate.

The term “pharmaceutically acceptable carrier” as used herein refers to any carrier, excipient, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well-known in the art. Except insofar as any conventional media or agent is incompatible with the active ingredient, its use in the therapeutic compositions is contemplated. Supplementary active ingredients can also be incorporated into the compositions as suitable therapeutic combinations.

The term “modulates” as used herein refers to a change in the level of activity, or the amount of, a constituent of an animal or human, an organ, thereof, or a cell or isolated cell thereof. For example, but not intended to be limiting, ‘modulate’ may refer to an increase in the amount of a protein such as IRS-2, thereby increasing the activity level of IRS-2 in a cell, tissue, or whole animal. Alternatively, “modulate” may refer to a decrease in the level of activity, or the amount of, a constituent of an animal or human, an organ, thereof, or a cell or isolated cell thereof.

The term “hypoxic response” as used herein refers to the response of a cell to conditions of low oxygen levels (hypoxia) to generate a hypoxia-related pathology. Hypoxia is a pathological condition in which the body as a whole (generalized hypoxia), a region of the body (tissue hypoxia), or the culturing conditions for isolated cells, is deprived of adequate oxygen supply. Variations in arterial oxygen concentrations can be part of the normal physiology, for example, during strenuous physical exercise. A mismatch between oxygen supply and its demand at the cellular level may result in a hypoxic condition in an animal. Hypoxia in which there is complete deprivation of oxygen supply is referred to as anoxia. The response may be generated, for example, by culturing the cells in the presence of prolyl hydroxylase inhibitors that act as hypoxia mimetics.

HIF is the primary transcription factor activated by hypoxia. Its activation and regulation are complex, with numerous points of potential inhibition. Active HIF is composed of alpha (HIF-1α, 2α) and beta (HIF-1β) subunits that dimerize and bind to consensus sequences (hypoxia responsive elements, HRE) in the regulatory regions of target genes. HIF controls the expression of more than 60 target genes, including, but not limited to, VEGF, erythropoietin, glucose transporters, and glycolytic enzymes. In normoxia, HIF is hydroxylated and interacts with the von Hippel Lindau protein (pVHL), an E3 ubiquitin ligase subunit that targets HIF for degradation. In the absence of oxygen, HIF hydroxylation is inhibited, preventing binding to pVHL and leading to its intracellular accumulation. The relative importance of HIF-1α and HIF-2α subunits in different tissues and cancer types is still under investigation as are their multiple levels of regulation. Accordingly, proline hydroxylase inhibitors reduce HIF hydroxylation, thus preventing HIF degradation, leading to HIF accumulation due to the increase in HIF stability.

Hydroxylation of HIF-α subunits can occur on proline and asparagine residues and can be mediated by a family of 2-oxoglutarate dependent enzymes. This family includes the HIF prolyl hydroxylase isozymes (PHDs), which hydroxylate Pro 402 and Pro 564 of human HIF1α, as well as Factor Inhibiting HIF (FIH), which hydroxylates Asn 803 of human HIF1α. Inhibition of FIH or the PHDs leads to HIF stabilization and transcriptional activation (see, for example, Schofield & Ratcliffe, (2004) Nature Rev. Mol. Cell. Biol. 5: 343-354).

The term “effective amount” as used herein refers to that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function

The term “selectively binds” as used herein refers the binding of one molecule to another, wherein the binding affinity between a first molecule, or ligand, and a second molecule is greater than the binding affinity of the ligand for a third molecule.

The term “hypoxia-related pathology” as used herein refers to a pathology that is caused in part, either directly or indirectly, by conditions of below typical physiological amounts of oxygen. The term “hypoxia-related pathology” also means a pathology caused by a non-hypoxic stimuli. The term includes cancer, cancer metastasis, ischemia, stroke and related conditions, diseases, or syndromes.

The terms “administration” or “administering” as used herein refers to a method of giving a dosage of a pharmaceutical composition to a subject, where the method is, but is not limited to, topical, transdermal, oral, intravenous, intraperitoneal, intracerebroventricular, intrathecal, or intramuscular. The preferred method of administration can vary depending on various factors, e.g. the various components of the pharmaceutical composition, site of administration, and severity of the symptoms being treated.

The terms “organism”, “host”, and “subject” as used herein refers to any living entity comprised of at least one cell. A living organism can be as simple as, for example, a single isolated eukaryotic cell or cultured cell or cell line, or as complex as a mammal, including a human being and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof.

The terms “treating” and “treatment” as used herein refer generally to obtaining a desired pharmacological and/or physiological effect. The effect may be prophylactic in terms of preventing or partially preventing a disease, symptom or condition thereof such as of diabetes, and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “treatment” as used herein particularly refers to the administration of a compound in an amount sufficient to, alleviate, ameliorate, or delay the progress of one or more symptoms or conditions associated with the disorder Type 2 diabetes mellitus.

### Description

The present disclosure encompasses methods of reversing the pathophysiological effects associated with Type 2 diabetes mellitus in an animal or human subject. In particular, the methods of the disclosure are directed to increasing the intracellular level of the hypoxia-induced protein HIF-2α with downstream and obligate increases in IRS-2 activity, accompanied by hepatic insulin sensitization. The present disclosure, therefore, provides methods of increasing HIF-2α and the concomitant increasing of the IRS-2 levels by blocking the interaction of the cell with VEGF, or by administering to the cells of the subject animal or human a composition inducing effects associated with hypoxia. It is contemplated, therefore, that the methods of the disclosure provide for the increase in IRS-2 activity in a target cell or tissue by administering to a subject animal or human an agent suitable for inhibiting VEGF availability for interaction with a VEGF receptor, or an agent that inhibits prolyl hydroxylation, thereby increasing HIF-2α levels.

By increasing the level of IRS-2 activity in the treated cells or tissues, and in particular in the hepatic cells of the animal or human subject, glucose synthesis by hepatic cells can be reduced, glycolysis can be increased, and serum glucose levels thereby reduced to approximately normal levels, even though the hepatic cells of the diabetic animal are non-responsive to serum insulin. By providing to the subject animal or human a composition that increases IRS-2 levels, the subject animal maintains hemostasis with respect to blood glucose when starved or fed high levels of glucose.

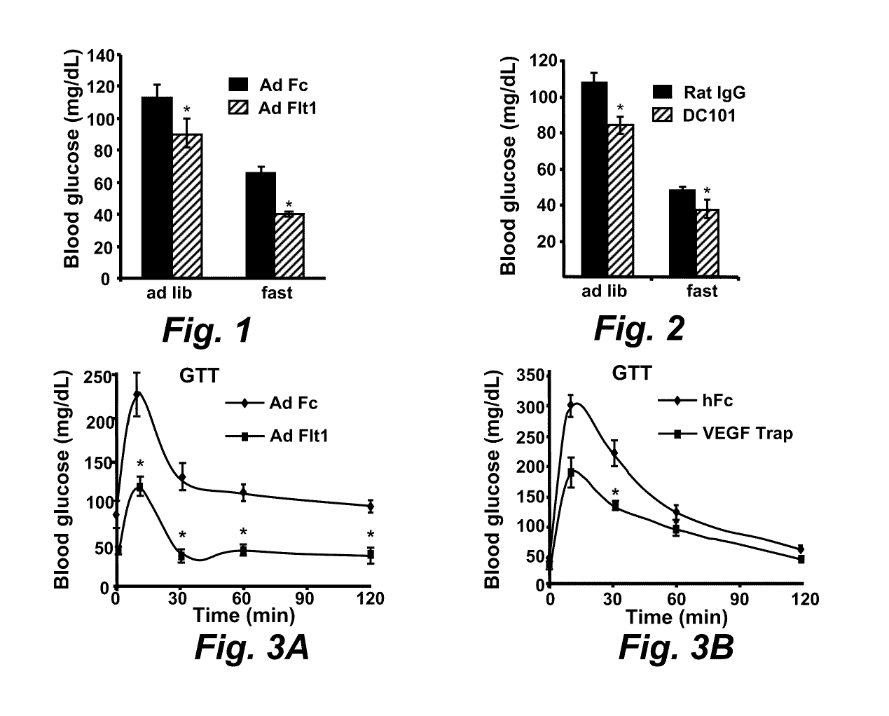

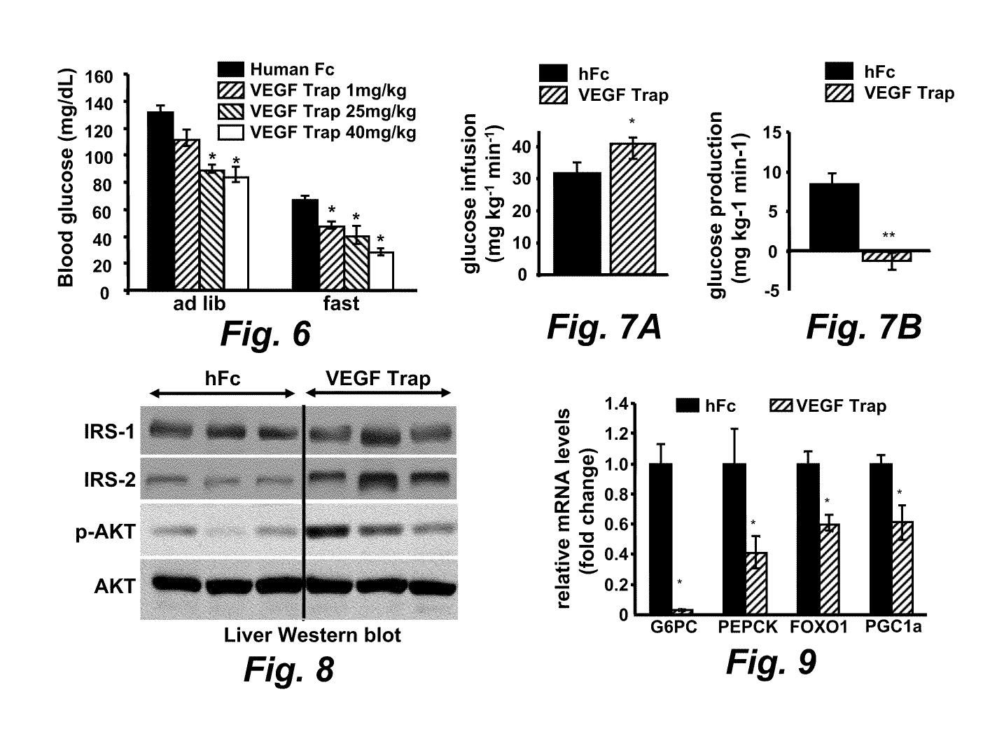

Accordingly, it has been found that both ad libidum and fasted blood glucose levels are lowered in mice treated with distinct VEGF inhibitors: adenovirus-mediated circulating expression of the soluble VEGF receptor Flt1 as used to obtain data of FIG. 1, for example, the VEGFR2-selective monoclonal antibody DC101 (as used to obtain data of FIG. 2), and by recombinant VEGF TRAP® (Regeneron Inc.), which is a soluble VEGF receptor fusion of the VEGF binding domains of Flk1 an Flt1 (as shown in FIG. 6). Glucose tolerance tests (GTT) also revealed improved glucose tolerance in these VEGF inhibitor-treated mice (FIGS. 3A and 3B).

The possibility of enhanced hepatic insulin signaling as the basis for decreased plasma glucose and enhanced glucose tolerance was investigated. Thus, Western blotting indicated increased hepatic insulin signaling following treatment with Ad Flt1 (VEGF inhibition), but not Ad Fc (negative control). (The adenovirus Flt1 (Ad Flt1) and hFc (Ad hFc) constructs were as described in Kuo et al (2001) Proc. Natl. Acad. Sci. USA 98: 4605-4610, incorporated herein by reference in its entirety).

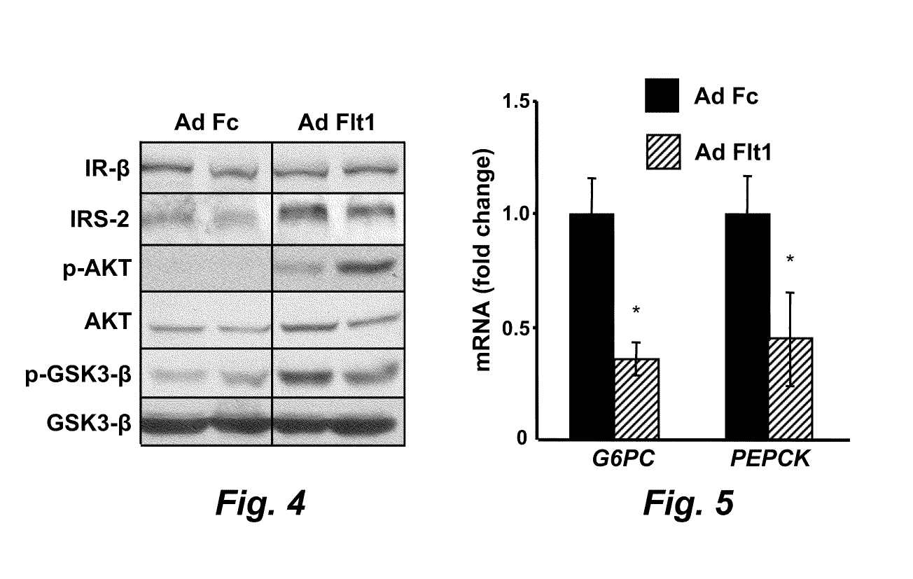

Ad Flt1, but not Ad Fc, increased phosphorylation of the insulin receptor (IR) signaling intermediates Akt (p-Akt) and GSK-3b (p-GSK-3b), as shown in FIG. 1D. Liver IRS-2 protein levels were also increased by Ad Flt1 (FIG. 4). Additionally, Ad Flt1 (VEGF inhibition) decreased expression of the hepatic gluconeogenic genes Pepck and G6P that undergo physiologic repression by insulin, consistent with enhanced hepatic insulin signaling upon VEGF inhibition, as shown in FIG. 5.

Hyperinsulinemic euglycemic clamp studies indicated that VEGF Trap-treated mice required a higher glucose infusion rate to maintain euglycemia and this was unequivocally attributable to increased insulin action in the liver (FIG. 7). Consistent with an insulin-sensitized state, livers from mice treated with VEGF Trap contained higher levels of IRS-2 and phospho-AKT, and hepatic expression of the glucose-6-phosphatase catalytic subunit (G6PC) and phosphoenolpyruvate carboxykinase (PEPCK) were significantly reduced (FIGS. 8-10).

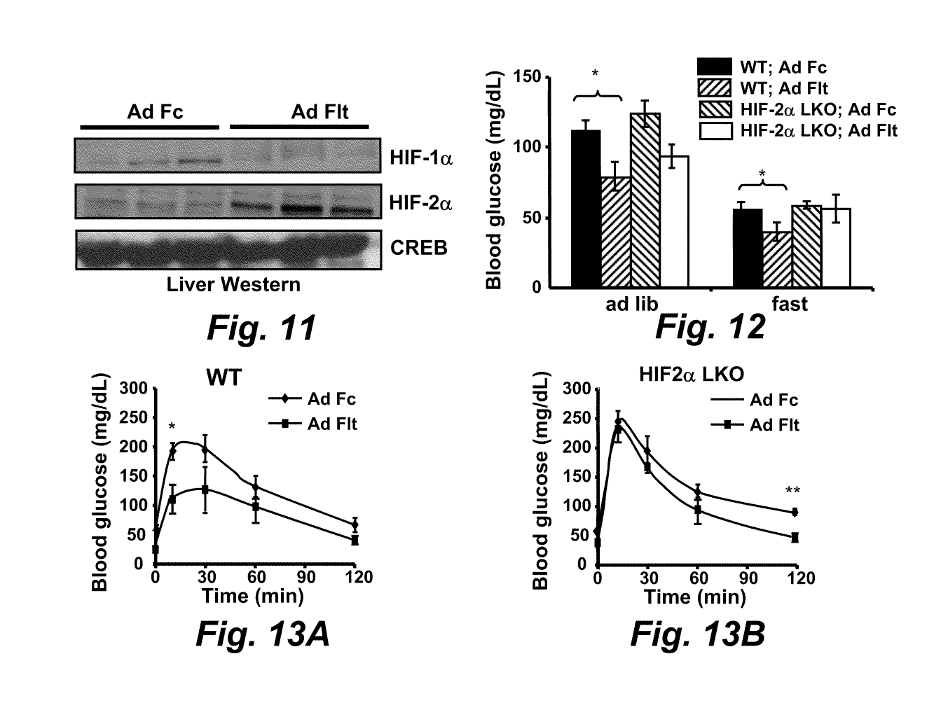

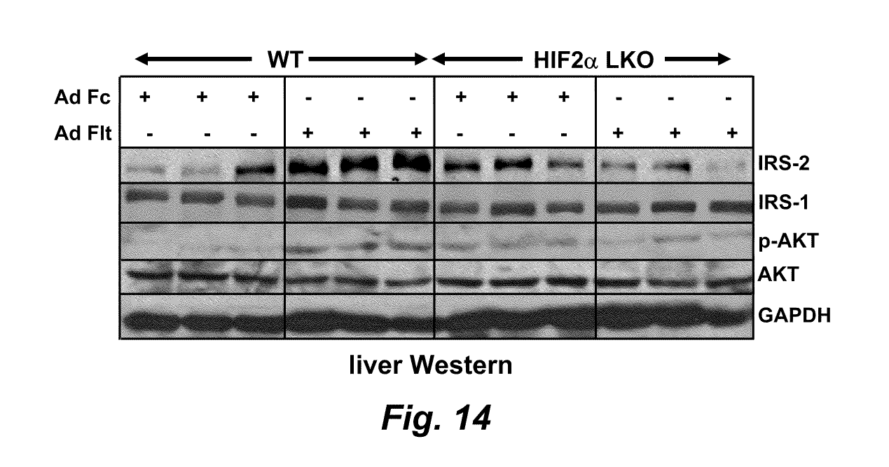

The mechanism by which VEGF-inhibition enhances hepatic insulin signaling was also investigated. There was increased stabilization of HIF-2α, but not of HIF-1α, in VEGF Trap-treated mice, as shown in FIG. 11. This stabilization of HIF-2α was functionally relevant since hepatocyte-specific deletion of HIF-2α (in HIf-2α^{flox/flox}; albumin-Cre mice) reversed the effects of VEGF inhibition on blood glucose level, glucose tolerance, and hepatic insulin sensitization, as shown in FIGS. 12-14), further indicating that hepatic HIF-2α is required to mediate the glucose metabolic response to VEGF inhibition.

Conversely, it was also determined if constitutive liver-specific activation of HIF-2α could be sufficient to phenocopy the effects of VEGF inhibition on blood glucose level, glucose tolerance, and hepatic insulin sensitization. Intravenous administration of adenovirus exhibited marked hepatotropism, allowing the attainment of constitutive liver-specific HIF-2α activation by adenoviral expression of a non-degradable HIF-2α (Ad HIF-2αPN, a double point mutant of human HIF2 with mutations at P5310A and Asp847Ala).

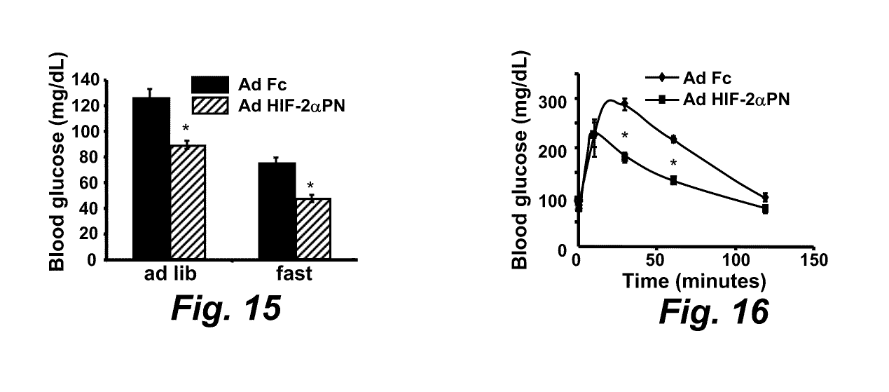

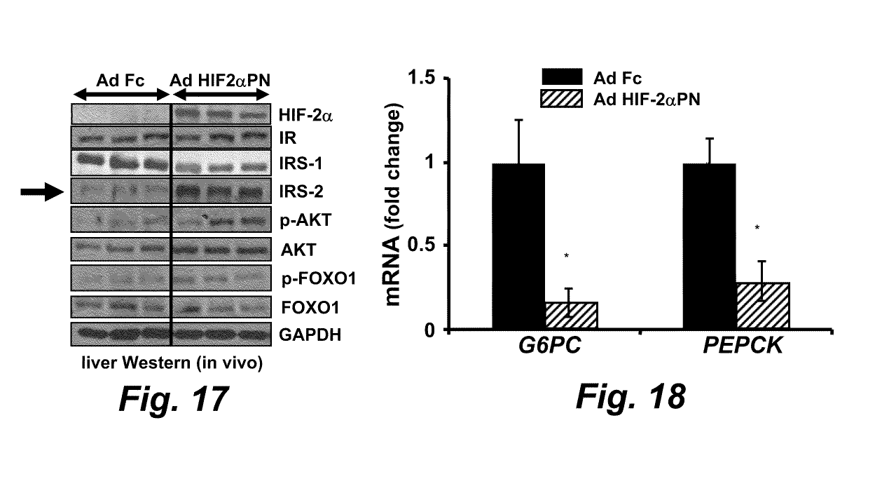

Ad HIF-2αPN reduced ad libidum and fasted blood glucose levels and improved glucose tolerance, as shown in FIGS. 15 and 16), as was also found with Ad Flt1 (see FIG. 1). Further, Ad HIF-2αPN was sufficient to activate downstream insulin signaling intermediates p-Akt and p-FOXO1 in liver extracts (as shown in FIG. 17), and to markedly repress the hepatic insulin-suppressed genes G6PC and PEPCK (as shown in FIG. 3D), again as with Ad Flt1 (see FIG. 1).

Ad HIF-2αPN also induces hepatic IRS-2 expression in vivo, as shown in FIG. 17), phenocopying the Ad Flt1 effect seen FIG. 1). Overall, the ability of the liver-specific expression of HIF-2αPN to phenocopy the effects of Ad Flt1 on blood glucose level, glucose tolerance, and sensitization of hepatic insulin signaling, as shown in FIG. 17, combined with the ability of Ad Flt1 to stabilize hepatic HIF-2α, as shown in FIG. 11, strongly supports that HIF-2α sensitization of liver insulin signaling can mediate blood glucose regulation by VEGF inhibitors and that HIF-2α stabilization, as could be achieved by hypoxia mimetics such as prolyl hydroxylase inhibitors and the like, is sufficient to improve glucose tolerance and sensitization of hepatic insulin signaling.

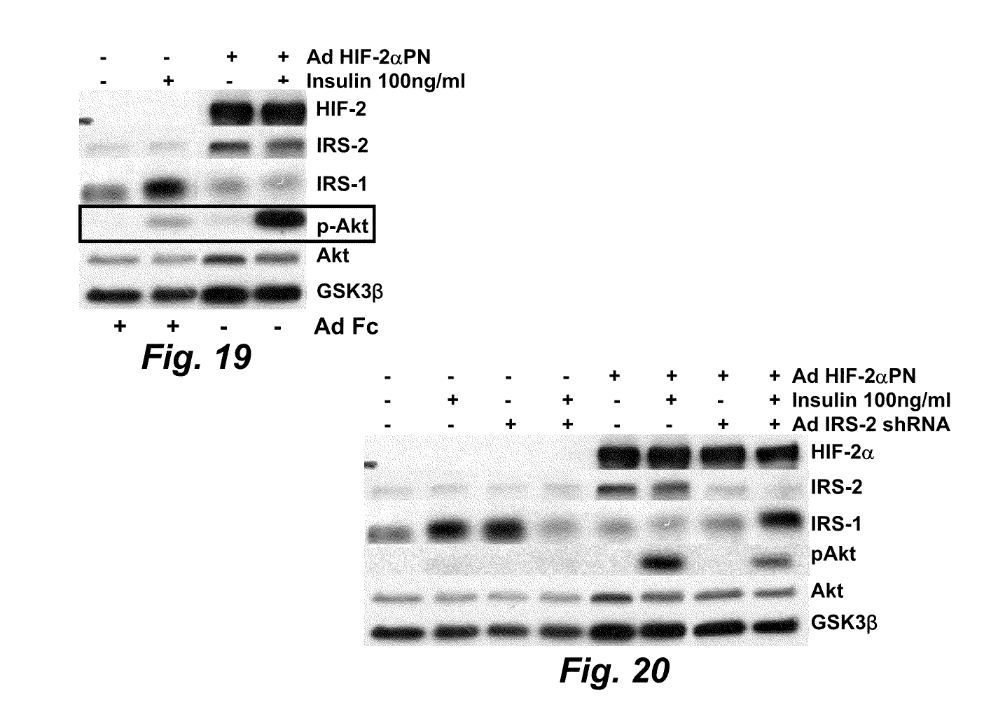

In vitro studies in cultured primary hepatocytes revealed identical regulation patterns, whereby HIF-2αPN and insulin synergized to induce Akt phosphorylation (p-Akt), showing that HIF-2α is sufficient to sensitize hepatocytes to insulin signaling in vitro (FIG. 19). HIF-2αPN strongly induced IRS-2 expression in cultured primary hepatocytes at the level of both protein and mRNA (FIGS. 19 and 20), paralleling in vivo observations (FIG. 17), indicating that HIF-2α stabilization, is sufficient to improve glucose tolerance, and to augment hepatic insulin signaling.

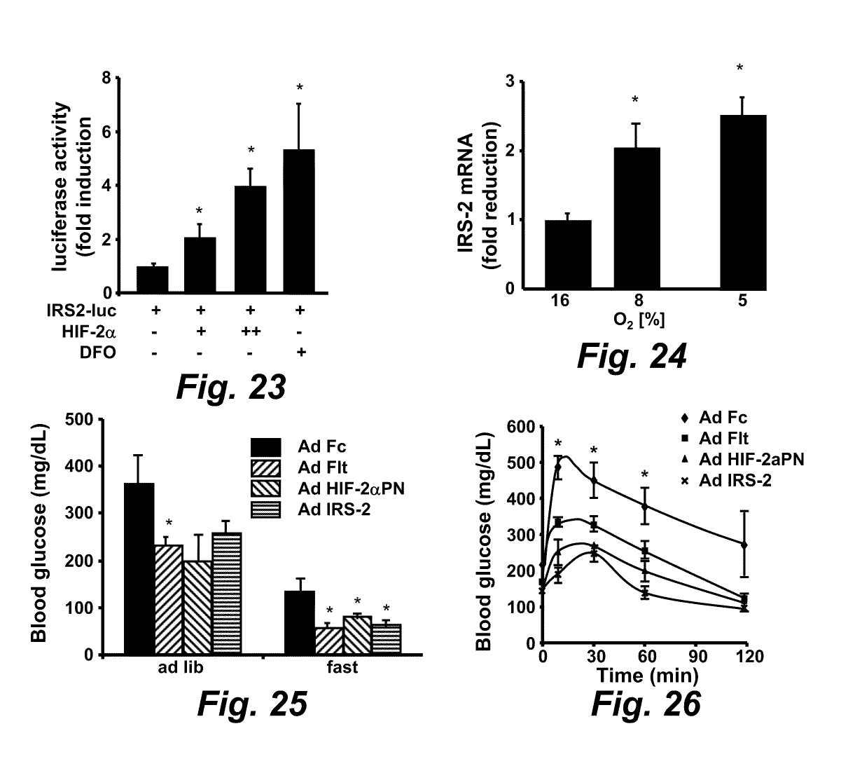

IRS-2 is a key downstream intermediate in the insulin signaling pathway. Accordingly, the synergistic promotion of Akt phosphorylation by Ad HIF-2αPN and insulin was blocked by IRS-2 shRNA, as shown in FIG. 20 (the nucleotide sequence of which is according to SEQ ID NO.: 1, as shown in Example 11, below). The synergistic activation of the insulin receptor (IR) signaling pathway by HIF-2α and insulin is schematized in FIG. 22, where HIF-2α sensitizes hepatocytes to insulin action. Additionally, IRS-2 was also shown to be hypoxia- and HIF-2α-inducible in cultured hepatocytes and 293T cells, indicating HIF-2α positively regulates hepatic insulin signaling through up-regulation of IRS-2 transcription (FIGS. 23 and 24).

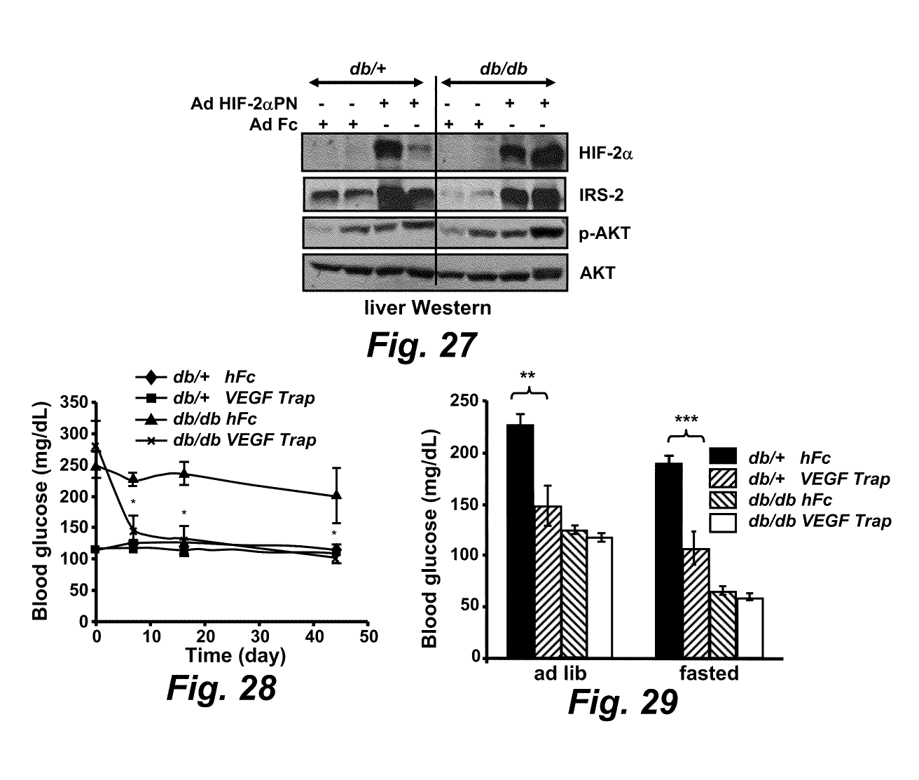

The ability of VEGF inhibitors to restore insulin sensitivity in db/db mice, a well-characterized mouse model of Type 2 diabetes mellitus, was also investigated. Treatment of db/db mice with Ad Flt1 significantly corrected hyperglycemia and improved glucose tolerance, as shown in FIGS. 25 and 26 and consistent with the data in wild-type mice (as shown in FIGS. 1 and 2). Liver-specific HIF-2α activation via Ad HIF-2αPN was sufficient to elicit an identical correction of db/db glucose levels and glucose intolerance, also as shown in FIGS. 25 and 26. Ad HIF-2αPN, but not Ad Fc control, restored IRS-2 expression to db/db liver, which is well recognized to be IRS-2-deficient (FIG. 27). In addition, restoration of IRS-2 to db/db liver via Ad IRS-2 was sufficient to correct the hyperglycemia and glucose intolerance of the db/db mouse (FIGS. 25 and 26). Overall, the robust correction of hyperglycemia and glucose intolerance of the db/db mouse by VEGF inhibition (i.e. by introducing Ad Flt1) indicated the potential utility of VEGF inhibitors for pharmacologic therapy of diabetes. Further, the ability of both Ad HIF-2αPN (i.e. HIF-2α activation) and Ad IRS-2 (i.e. restoration of IRS-2 to the IRS-2-deficient db/db liver) to phenocopy VEGF inhibitor treatment in db/db mice strongly supported that a VEGF inhibitor→HIF-2α→IRS-2 axis which is a suitable target for the manipulation for diabetes therapy and also for the screening of potential therapeutic agents suitable for use as a diabetes treatment. These results also indicate that HIF-2α stabilization, as could be achieved by hypoxia mimetics such as prolyl hydroxylase inhibitors and the like, is sufficient to improve glucose tolerance and to correct diabetes.

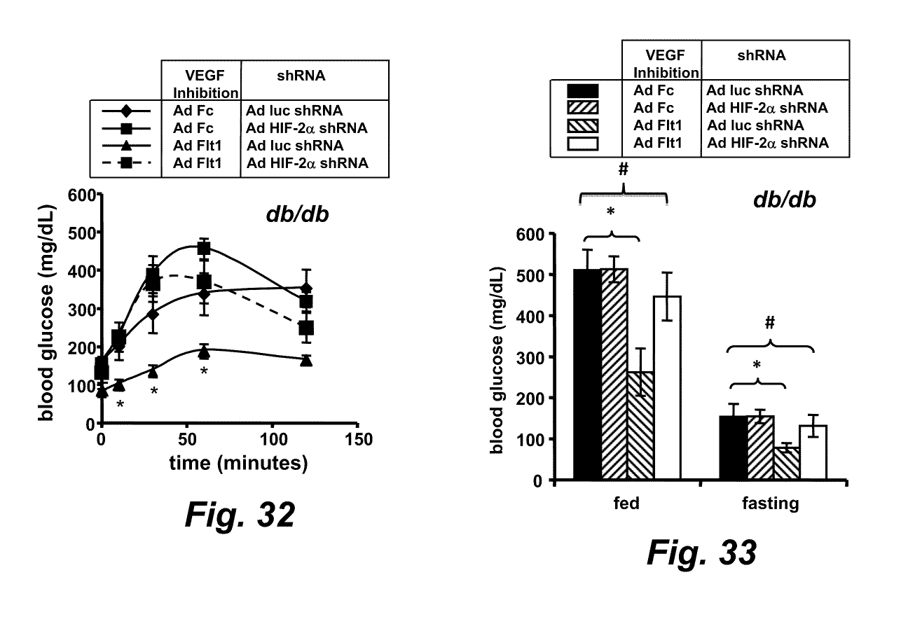

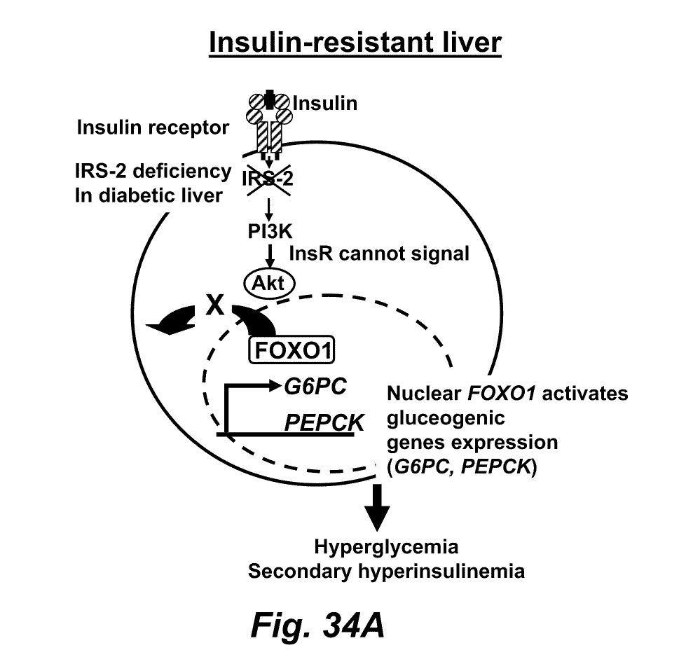

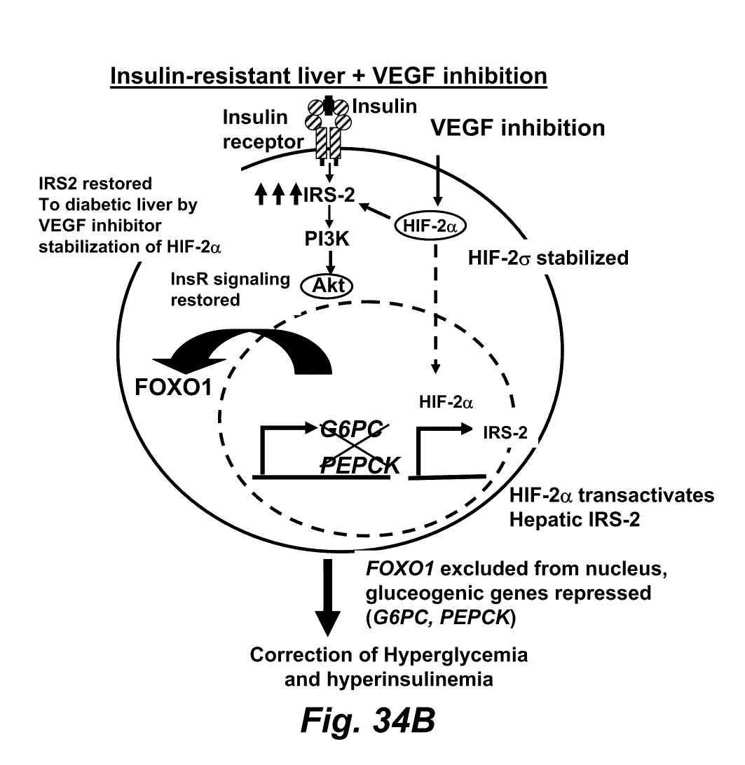

To further prove the functional existence of this pathway, HIF-2α inhibition via Ad HIF-2α shRNA (SEQ ID NO.: 1) was used to block the therapeutic effects of VEGF inhibition in the db/db mouse model. Accordingly, it was found that Ad HIF-2α shRNA strongly inhibited the effects of Ad Flt1 on hyperglycemia and glucose tolerance in db/db mice, as shown in FIGS. 32 and 33), indicating an essential role of hepatic HIF-2α downstream of any VEGF inhibitor activity. Long-term administration of VEGF Trap in db/db mice led to sustained correction of hyperglycemia and hyperinsulinemia, and improved glucose tolerance (FIGS. 28-31). These results indicate that activation of hepatic HIF-2α signaling is sufficient to sensitize hepatic insulin signaling and recapitulate the hepatic glucose metabolic response to VEGF inhibition.

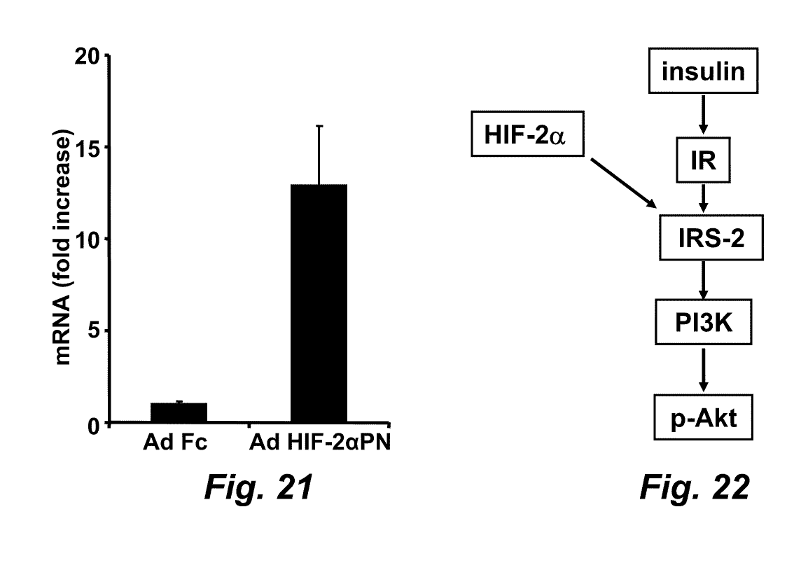

While not wishing to be bound by any one theory, the data shown in FIGS. 1-33 support that (i) VEGF inhibition robustly corrects hyperglycemia and hyperinsulinemia in Type 2 diabetes mellitus models, and that (ii) this correction occurs through a novel hepatic HIF-2α-IRS-2 axis. These data also (iii) unexpectedly unify hypoxia-induced gene expression and insulin receptor signaling, (iv) expand the repertoire of hypoxia effects on metabolism beyond glucose transport, and (v) the utility of VEGF inhibitors for diabetes treatment, as well as the use of hypoxia-mimetic small molecule prolyl hydroxylase inhibitors that stabilize HIFs and which are already in clinical trials, as suitable therapeutic agents for the reduction of blood glucose levels in animals and therefore the suitability as therapeutic agents for the treatment of type II diabetes.

One aspect of the disclosure provides embodiments of a method of modulating the level of glucose metabolism of a mammalian cell, comprising: contacting a mammalian cell with an effective amount of a composition, where the composition can modulate the levels of HIF-2α and IRS-2 activity in said cell, thereby modulating the level of glucose metabolism by said cell.

In embodiments of this aspect of the disclosure, the modulation of the HIF-2α level in the mammalian cell increases the level of IRS-2 activity in the cell, thereby decreasing the level of activity of at least one gluconeogenic enzyme of the cell.

In embodiments of this aspect of the disclosure, the modulation of the HIF-2α level in the mammalian cell increases the level of IRS-2 activity in the cell, thereby increasing the level of activity of at least one glycolytic enzyme of the cell.

In embodiments of this aspect of the disclosure, an increase in HIF-2α activity in the mammalian cell can increase the level of IRS-2 activity in the cell.

In some embodiments of this aspect of the disclosure, the composition modulating the level of HIF-2α in the cell modulates the interaction of VEGF with the cell.

In some embodiments of this aspect of the disclosure, the composition reduces the interaction of VEGF and the cell compared to when the composition is not in contact with the cell.

In embodiments of this aspect of the disclosure, the composition is a VEGF antagonist. In these embodiments of this aspect of the disclosure, the composition can be a VEGF antagonist selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In other embodiments of this aspect of the disclosure, the composition generates an hypoxic response mimetic in the mammalian cell, thereby increasing the level of HIF-2α in the cell.

In embodiments of this aspect of the disclosure, the mammalian cell can be an hepatic cell.

In embodiments of this aspect of the disclosure, the mammalian cell can be an isolated cell or a population thereof.

In embodiments of this aspect of the disclosure, the mammalian cell can be in a tissue of an animal or human, and wherein the level of glucose in the blood of the recipient animal or human is reduced.

Another aspect of the disclosure provides embodiments of a method for the treatment of Type 2 diabetes in an animal or human subject, the method comprising administering to an animal or human subject a effective amount of a pharmaceutically acceptable composition, the composition, when administered to an animal or human subject, increasing the activity of HIF-2α and IRS-2 in the animal or human subject, thereby reducing the level of blood glucose in the animal or human subject.

In embodiments of this aspect of the disclosure, the pharmaceutically acceptable composition comprises an antagonist of VEGF activity in the subject.

In these embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In some embodiments of this aspect of the disclosure, the pharmaceutically acceptable composition can generate an hypoxic response mimetic in the subject animal or human and comprises an inhibitor of a prolyl hydroxylase activity.

In embodiments of this aspect of the disclosure, the modulation of the HIF-2α level in the cell can increase the level of IRS-2 activity, thereby decreasing the level of activity of at least one gluconeogenic enzyme.

In these and other embodiments of this aspect of the disclosure, the modulation of the HIF-2a level in the cell increases the level of IRS-2 activity, thereby increasing the level of activity of at least one glycolytic enzyme.

Still another aspect of the disclosure provides embodiments of a pharmaceutical composition comprising an effective dose of a therapeutic agent that when administered to a animal or human subject induces an increase in the level of HIF-2α activity in the cell, thereby decreasing the blood glucose level of the recipient subject.

In embodiments of this aspect of the disclosure, the pharmaceutical composition, when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

In embodiments of this aspect of the disclosure, the therapeutic agent can be a VEGF antagonist in an amount that when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

In embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

In other embodiments of this aspect of the disclosure the therapeutic agent can generate an hypoxic response mimetic in the subject animal or human and comprises an inhibitor of a prolyl hydroxylase activity.

Yet another aspect of the disclosure provides embodiments of an article of manufacture comprising a package containing a pharmaceutical composition that when administered as an effective dose to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased, where the pharmaceutical composition can comprise a therapeutic agent that is a VEGF antagonist or an hypoxic response mimetic, said mimetic comprising an inhibitor of a prolyl hydroxylase activity.

In embodiments of this aspect of the disclosure, the VEGF antagonist can be selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### Examples

### Example 1

Tail Biopsies: A 1 cm or smaller portion of the tip of the mouse tail was excised between the ages of 6-12 days using aseptic techniques.

Adenovirus injection: Adult wild-type, db/db, or HIF-2α^{φlox/flox}; alb-Cre mice were placed into a postural restraint followed by single intravenous tail vein injection of 10⁹ pfu of adenovirus encoding inserts such as Cre, Flt1, HIF-2α^{φ}shRNA, or IRS-2 shRNA in PBS/3% glycerol using a 30 G needle.

Administration of recombinant VEGF inhibitors: Animals were anesthetized with methoxyflurane followed by subcutaneous injection of DC101 or control antibody in PBS in the flank.

Mice were anesthetized with methoxyflurane inhalation (bell jar effect) or pentobarbital (10-20 mg/kg intraperitoneally) before procedures. Local anesthesia was assessed by the pinch method and respiratory rate. After procedure completion, animals are observed until normal mobility was recovered and homeostasis attained. Experimental animals were monitored daily by gross observations. Any animal judged incapable of performing the necessary functions of life including ability to eat, sleep, and move about independently in the cage would be considered a failure and would be sacrificed by CO₂ asphyxiation.

### Example 2

### Role of HIF-2α.

Db/db model. Db/db or wild-type mice received single intravenous injection of adenoviruses encoding Ad Flt (soluble VEGF receptor), Ad Fc (control antibody Fc fragment), VEGF Trap or control human Fc fragment, with or without adenovirus encoding shRNA against HIF-2α, or scrambled control shRNA. Adenovirus gives preferential and quantitative hepatocyte infection, a robust maneuver for achieving hepatic gene knockdown (Canettieri et al., (2005) Cell Metab. 2: 331; Taniguchi et al., (2005) J. Clin. Invest. 115: 718).

### Example 3

FIGS. 1-9 show a series of graphs and digital images indicating glucose homeostasis and augmented hepatic insulin signaling in VEGF inhibitor-treated wild-type mice.

### Example 4

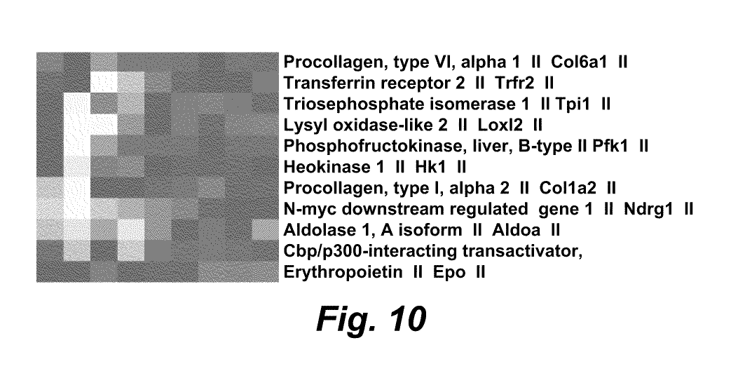

FIGS. 10-14 illustrate that the metabolic glucose response to VEGF inhibition requires hepatic HIF-2α.

### Example 5

FIGS. 15-16 show a series of graphs showing that HIF-2α is required for VEGF blockade-induced glucose metabolism and hepatic insulin signaling in 7-8 week-old HIF-2α^{loxP/loxP} (i.e. wild-type) and HIF-2α^{loxP/loxP}; albumin-Cre mice (i.e., liver-specific knockout “LKO” of HIF-2α) (n=8) injected with 5×10⁸ pfu Ad Flt1 or Ad Fc intravenously.

### Example 6

FIGS. 15-20 illustrate that liver-specific HIF-2α activation is sufficient to up-regulate hepatic IRS-2 and insulin signaling in parallel in adult 10-12 week-old C57Bl/6 (n=5) mice receiving single intravenous injections of 1×10⁸ pfu Ad HIF-2αPN (i.e. constitutively active mutant form of HIF-2α) or Ad Fc (negative control).

### Example 7

FIGS. 19-24 illustrate that HIF-2α activation is sufficient to up-regulate hepatocyte insulin signaling in an IRS-2-dependent manner in vitro.

### Example 8

FIGS. 25-27 illustrate that VEGF inhibition and HIF-2α activation reverse the diabetic phenotype of db/db mice. The data of FIGS. 5A-5C are from 10 week-old female db/db mice injected with 5×10⁸ pfu of the indicated viruses. These data indicate that VEGF inhibition or HIF-2α stabilization, as could also be achieved by hypoxia mimetics such as prolyl hydroxylase inhibitors and the like, is sufficient to improve glucose tolerance and reverse diabetic phenotypes.

### Example 9

FIGS. 28-29 illustrate that the chronic administration of VEGF Trap protein improves hepatic glucose tolerance in db/db mice. 10 week-old female db/db and db/+ mice (n=5) were treated with twice-weekly subcutaneous injections of 25 mg/kg VEGF Trap or control hFc.

### Example 10

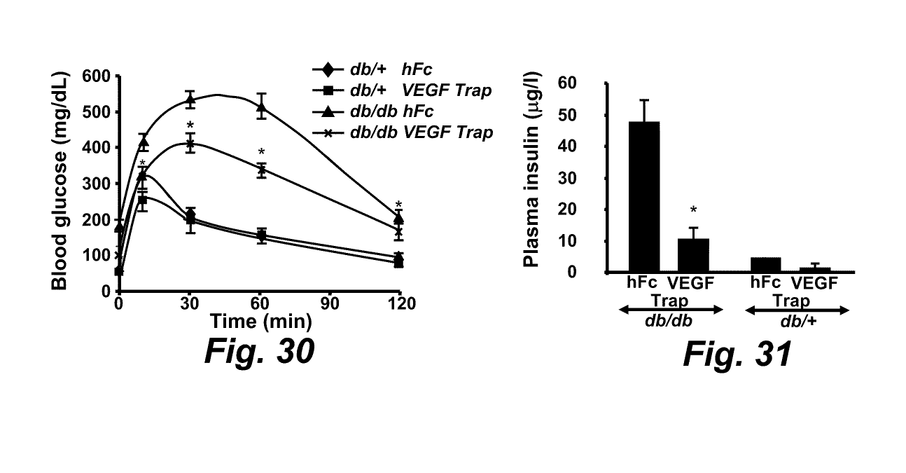

FIGS. 30-33 illustrate that VEGF inhibitors require HIF-2α to correct glucose intolerance in db/db mice. The data of FIGS. 31 and 32 are from 10 week-old female db/db mice injected with 1×10⁸ pfu of Ad Flt1/Ad Fc and 5×10⁸ pfu of Ad HIF-2α shRNA/Ad luc shRNA. Ad luc shRNA expresses a negative control luciferase shRNA.

### Example 11

Nucleotide sequence of shRNA SEQ ID No.: 1:
5′-TGCUGUUGACAGUGAGCGAGGAGCUUAACUUUAUAAGGAAUAGUGAA
GCCACAGAUGUAUUCCUUAUAAAGUUAAGCUCCCUGCCUACUGCCUCGG
A-3′

1. A method of modulating the level of glucose metabolism of a mammalian cell, comprising:
contacting a mammalian cell with an effective amount of a composition, wherein the composition modulates the levels of HIF-2α and IRS-2 activity in said cell, thereby modulating the level of glucose metabolism by said cell.

2. The method according to claim 1, wherein the modulation of the HIF-2α level in the mammalian cell increases the level of IRS-2 activity in the cell, thereby decreasing the level of activity of at least one gluconeogenic enzyme of the cell.

3. The method according to claim 1, wherein the modulation of the HIF-2a level in the mammalian cell increases the level of IRS-2 activity in the cell, thereby increasing the level of activity of at least one glycolytic enzyme of the cell.

4. The method according to claim 1, wherein an increase in HIF-2α activity in the mammalian cell increases the level of IRS-2 activity in the cell.

5. The method according to claim 1, wherein the composition modulating the level of HIF-2α in the cell modulates the interaction of VEGF with the cell.

6. The method according to claim 5, wherein the composition reduces the interaction of VEGF and the cell compared to when the composition is not in contact with the cell.

7. The method according to claim 6, wherein the composition is a VEGF antagonist.

8. The method according to claim 7, wherein the composition is a VEGF antagonist selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

9. The method according to claim 1, wherein the composition generates an hypoxic response mimetic in the mammalian cell, thereby increasing the level of HIF-2α in the cell.

10. The method according to claim 1, wherein the mammalian cell is an hepatic cell.

11. The method of claim 7, wherein the mammalian cell is an isolated cell or a population thereof.

12. The method of claim 1, wherein the mammalian cell is in a tissue of an animal or human, and wherein the level of glucose in the blood of the recipient animal or human is reduced.

13. A method for the treatment of Type 2 diabetes in an animal or human subject, the method comprising administering to an animal or human subject a effective amount of a pharmaceutically acceptable composition, wherein said composition, when administered to an animal or human subject, increases the activity of HIF-2α and IRS-2 in the animal or human subject, thereby reducing the level of blood glucose in the animal or human subject.

14. The method according to claim 13, wherein the pharmaceutically acceptable composition comprises an antagonist of VEGF activity in the subject.

15. The method according to claim 14, wherein the VEGF antagonist is selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

16. The method according to claim 13, wherein the pharmaceutically acceptable composition generates an hypoxic response mimetic in the subject animal or human and comprises an inhibitor of a prolyl hydroxylase activity.

17. The method according to claim 13, wherein the modulation of the HIF-2α level in the cell increases the level of IRS-2 activity, thereby decreasing the level of activity of at least one gluconeogenic enzyme.

18. The method according to claim 13, wherein the modulation of the HIF-2a level in the cell increases the level of IRS-2 activity, thereby increasing the level of activity of at least one glycolytic enzyme.

19. A pharmaceutical composition comprising an effective dose of a therapeutic agent that when administered to a animal or human subject induces an increase in the level of HIF-2α activity in the cell, thereby decreasing the blood glucose level of the recipient subject.

20. The pharmaceutical composition of claim 19 that when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

21. The pharmaceutical composition of claim 19, wherein the therapeutic agent is a VEGF antagonist in an amount that when administered to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased.

22. The pharmaceutical composition of claim 21, wherein the VEGF antagonist is selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.

23. The pharmaceutical composition of claim 19, wherein the therapeutic agent generates an hypoxic response mimetic in the subject animal or human and comprises an inhibitor of a prolyl hydroxylase activity.

24. The pharmaceutical composition of claim 19 further comprising a pharmaceutically acceptable carrier.

25. An article of manufacture comprising a package containing a pharmaceutical composition that when administered as an effective dose to an animal or human subject in need thereof, increases the HIF-2α level in the recipient subject and increases the level of IRS-2 activity, thereby decreasing the level of activity of at least one gluconeogenic enzyme or increasing the level of activity of at least one glycolytic enzyme, or a combination thereof, whereby the level of blood glucose of the subject is decreased, wherein the pharmaceutical composition comprises a therapeutic agent that is a VEGF antagonist or an hypoxic response mimetic, said mimetic comprising an inhibitor of a prolyl hydroxylase activity.

26. The article of manufacture of claim 25, wherein the VEGF antagonist is selected from the group consisting of: an anti-VEGF antibody or fragment thereof, an anti-VEGF receptor antibody or fragment thereof, a soluble VEGF receptor variant, a ligand-binding fragment of a VEGF receptor, a small molecule kinase inhibitor, nucleic acid aptamer, an siRNA, and an shRNA, wherein the VEGF antagonist is characterized as selectively binding to VEGF or to a VEGF receptor, or interferes with VEGF signaling.


SEQUENCE LISTING (1 sequence)

SEQ ID NO: 1 - RNA, 97 nt, Artificial sequence
  shRNA sequence
ugcuguugacagugagcgaggagcuuaacuuuauaaggaauagugaagccacagauguauuccuuauaaaguuaagcucccugccuacugccucgga